SEQUENCE LISTING (27 sequences)

SEQ ID NO: 1 - protein, 40 aa, Artificial Sequence
  misc_feature: PTHrP - (1-40)
AVSEHQLLHDKGKSIQDLRRRFFLHHLIAEIHTAEIRATS

SEQ ID NO: 2 - protein, 84 aa, Bos sp.
  misc_feature: bPTH
AVSEIQFMHNLGKHLSSMERVEWLRKKLQDVHNFVALGASIAYRDGSSQRPRKKEDNVLVESHQKSLGEADKADVDVLIKAKPQ

SEQ ID NO: 3 - protein, 22 aa, Artificial Sequence
  misc_feature: [Tyr63]-hPTH (63-84)
YEKSLGEADKADVNVLTKAKSQ

SEQ ID NO: 4 - protein, 21 aa, Homo sapiens
  misc_feature: hPTH (64-84)
EKSLGEADKADVNVLTKAKSQ

SEQ ID NO: 5 - protein, 16 aa, Artificial Sequence
  misc_feature: [Tyr69]-hPTH (69-84)
YADKADVNVLTKAKSQ

SEQ ID NO: 6 - protein, 15 aa, Homo sapiens
  misc_feature: hPTH (70-84)
ADKADVNVLTKAKSQ

SEQ ID NO: 7 - protein, 13 aa, Homo sapiens
  misc_feature: Human Bone Gla Protein (37-49) (BGP 37-49)
GFQEAYRRFYGPV

SEQ ID NO: 8 - protein, 12 aa, Artificial Sequence
  misc_feature: [Tyr36 , Phe42,46 ]-Human Bone Gla Protein (38- 49)
YQEAFRRFFGPV

SEQ ID NO: 9 - protein, 5 aa, Homo sapiens
  misc_feature: Human Bone Gla Protein (45-49)
FYGPV

SEQ ID NO: 10 - protein, 84 aa, Homo sapiens
  misc_feature: hPTH
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 11 - protein, 84 aa, Rattus sp.
  misc_feature: Rat PTH (rPTH)
AVSEIQLMHNLGKHLASVERMQWLRKKLQDVHNFVSLGVQMAAREGSYQRPTKKEDNVLVDGNSKSLGEGDKADVDVLVKAKSQ

SEQ ID NO: 12 - protein, 34 aa, Bos sp.
  misc_feature: bPTH (1-34)
AVSEIQFMHNLGKHLSSMERVEWLRKKLQDVHNF

SEQ ID NO: 13 - protein, 34 aa, Homo sapiens
  misc_feature: hPTH (1-34)
SVSEIQLMHNLGKHLNSMERVEWLRKKLQDVHNF

SEQ ID NO: 14 - protein, 34 aa, Artificial Sequence
  MOD_RES (8)..(8): Nle
  MOD_RES (18)..(18): Nle
  misc_feature: [Nle8,18, Tyr34]-bPTH (1-34)
AVSEIQFXHNLGKHLSSXERVEWLRKKLQDVHNY

SEQ ID NO: 15 - protein, 34 aa, Artificial Sequence
  MOD_RES (8)..(8): Nle
  MOD_RES (18)..(18): Nle
  misc_feature: [Nle8,18, Tyr34] hPTH (1-34)
SVSEIQLXHNLGKHLNSXERVEWLRKKLQDVHNY

SEQ ID NO: 16 - protein, 34 aa, Artificial Sequence
  MOD_RES (8)..(8): Nle
  MOD_RES (21)..(21): Nle
  misc_feature: [Nle8,21, Tyr34]-rPTH (1-34)
AVSEIQLXHNLGKHLASVERXQWLRKKLQDVHNY

SEQ ID NO: 17 - protein, 34 aa, Artificial Sequence
  misc_feature: [Tyr1]-hPTH (1-34)
YVSEIQLMHNLGKHLNSMERVEWLRKKLQDVHNF

SEQ ID NO: 18 - protein, 38 aa, Homo sapiens
  misc_feature: hPTH (1-38)
SVSEIQLMHNLGKHLNSMERVEWLRKKLQDVHNFVALG

SEQ ID NO: 19 - protein, 44 aa, Homo sapiens
  misc_feature: hPTH (1-44)
SVSEIQLMHNLGKHLNSMERVEWLRKKLQDVHNFVALGAPLAPR

SEQ ID NO: 20 - protein, 32 aa, Bos sp.
  misc_feature: bPTH (3-34)
SEIQFMHNLGKHLSSMERVEWLRKKLQDVHNF

SEQ ID NO: 21 - protein, 32 aa, Artificial Sequence
  MOD_RES (6)..(6): Nle
  MOD_RES (16)..(16): Nle
  misc_feature: [Nle8,18, Tyr34]-bPTH (3-34)
SEIQFXHNLGKHLSSXERVEWLRKKLQDVHNY

SEQ ID NO: 22 - protein, 28 aa, Artificial Sequence
  MOD_RES (2)..(2): Nle
  MOD_RES (12)..(12): Nle
  misc_feature: [Nle8,18, Tyr34]-bPTH (7-34)
FXHNLGKHLSSXERVEWLRKKLQDVHNY

SEQ ID NO: 23 - protein, 28 aa, Artificial Sequence
  misc_feature: [Tyr34]-bPTH (7-34)
FMHNLGKHLSSMERVEWLRKKLQDVHNY

SEQ ID NO: 24 - protein, 22 aa, Homo sapiens
  misc_feature: hPTH (13-34)
KHLNSMERVEWLRKKLQDVHNF

SEQ ID NO: 25 - protein, 22 aa, Artificial Sequence
  misc_feature: [Tyr27]-hPTH (27-48)
YLQDVHNFVALGAPLAPRDAGS

SEQ ID NO: 26 - protein, 21 aa, Homo sapiens
  misc_feature: hPTH (28-48)
LQDVHNFVALGAPLAPRDAGS

SEQ ID NO: 27 - protein, 32 aa, Homo sapiens
  misc_feature: hPTH (53-84)
KKEDNVLVESHEKSLGEADKADVNVLTKAKSQ

(19) United States
(12) Patent Application Publication
(10) Pub. No.: US 2007/0128265 A1
(43) Pub. Date: Jun. 7, 2007

(54) Regulation of cell proliferation and differentiation using topically applied peptides

(76) Inventor: Michael F. Holick, Sudbury, MA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/639,238

(22) Filed: Dec. 15, 2006

Related U.S. Application Data
(63) Continuation of application No. 10/311,366, filed on May 28, 2003, now abandoned.
(60) Provisional application No. 60/213,247, filed on Jun. 22, 2000.

(51) Int. Cl. A61K 38/39; A61K 31/59; A61K 9/127

(52) U.S. Cl. 424/450; 514/12; 514/167

(57) ABSTRACT

Methods are disclosed for the regulation of cell differentiation and proliferation, e.g., for treating hyperproliferative skin disorders, such as psoriasis, for enhancing wound healing, for stimulating hair growth and inhibiting hair growth, by topical administration of parathyroid hormone (PTH), parathyroid related peptide (PTHrP), or fragment, analog or derivative thereof, and salts thereof, encapsulated by liposomes.

### BACKGROUND OF THE INVENTION

1. Field of the Invention

This invention relates to the regulation of cell differentiation and proliferation, e.g., for treating hyperproliferative skin disorder, such as psoriasis, for enhancing wound healing, for stimulating hair growth, and inhibiting hair growth by topical administration of parathyroid hormone (PTH), parathyroid related peptide (PTHrP), or fragment, analog or derivative thereof, and salts thereof, encapsulated by particular liposomes.

2. Related Art

U.S. Pat. Nos. 5,527,772, 5,840,690 and 6,066,618 describe methods of inhibiting proliferation and enhancing differentiation of mammalian cells, inducing proliferation of mammalian cells, enhancing wound healing, and stimulating hair growth using a peptide which has a 10% or greater homology to a region of human PTH or human PTHrP. Certain fragments and analogs (e.g. PTH (1-34), PTH (3-34) and PTHrP (1-34)) were found to act as agonists of PTH and PTHrP and inhibit proliferation and enhance differentiation of mammalian cells. Other fragments and analogs (e.g. PTH (7-34) and PTHrP (7-34) are antagonists of PTH and PTHrP and enhance the proliferation of mammalian cells. The agonists are useful for treatment of hyperproliferative skin diseases such a psoriasis and the antagonists are useful for wound healing, particularly wounds of the skin, enhancing or maintaining hair growth, particularly following chemotherapeutic treatment of a mammal, and stimulating epidermal regrowth. Methods of administration include oral, nasal, intravenous, subcutaneous, parenteral and intraperitoneal administration. The peptides may be administered by subcutaneous pumps, patches, tapes, or by liposomal carriers.

A variety of PTH and PTHrP analogs and derivatives thereof have been made. See U.S. Pat. Nos. 4,086,196, 4,423,037, 4,771,124, 4,833,125, 4,968,669, 5,001,223, 5,087,562, 5,093,233, 5,116,952, 5,149,779, 5,171,670, 5,229,489, 5,317,010, 5,382,658, 5,393,869, 5,434,246, 5,527,772, 5,589,452, 5,807,823, 5,821,255, 5,840,690, 5,977,070, 6,025,467, 6,051,868, and 6,066,618; WO094/02510, WO00/23594, and WO00/31137; and EP 477,885. Methods for determining whether a particular analog is an agonist or antagonist of PTH and PTHP are described in U.S. Pat. Nos. 5,527,772, 5,840,690 and 6,066,618.

Active vitamin D compounds are useful for treating hyperproliferative skin diseases and other conditions. A large number of such active vitamin D compounds are known. See U.S. Pat. Nos. 5,457,217, 5,414,098, 5,384,313, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,857;518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

### SUMMARY OF THE INVENTION

The invention provides two important therapeutic methods one involving inhibition of cell proliferation and enhancement of skin cell differentiation (the agonist activity), which is useful in the treatment of psoriasis, ichthyosis, actinic keratoses, skin cancer, inhibiting hair growth or preventing hair regrowth. A second method involves enhancement of cell proliferation (the antagonist activity), which is useful in wound healing, stimulating epidermal regrowth and hair growth. In addition, the invention provides methods for enhancing wound healing and hair growth based on in vivo wound healing activity or in vitro or in vivo hair growth activity rather than strict agonist or antagonist activity in vitro.

The first method of the invention generally involves inhibiting proliferation and enhancing differentiation of mammalian skin cells by contacting the cell with a liposomal preparation comprising a peptide (preferably at least 3, and more preferably at least 8, amino acids long) which has 10% or greater (more preferably, 50% or greater, and most preferably 75% or greater) sequence identity with a region (preferably within the amino-terminal 34 amino acid region) of human PTH or human PTHrP, and which is capable of inhibiting proliferation or enhancing the differentiation in vitro of cultured human keratinocytes; or in vivo in mouse skin by inhibiting skin cell proliferation or hair cycle progression or hair growth. In preferred embodiments of this method, the peptide is hPTH (1-84), hPTH (1-34), hPTH (3-34), hPTHrP (1-34), hPTHrP (1-141), hPTHrP (1-139) or hPTHrP (1-173). This method has particular application in the treatment of hyperproliferative skin disorders such as psoriasis. The method may also be useful in the treatment of certain skin cancers, by the inhibition of cancer cell proliferation and by the induction of differentiation and inhibition of hair growth.

The second method of the invention generally involves enhancing proliferation of mammalian skin cells by contacting the skin cells with a liposomal preparation comprising a peptide (preferably at least 3, and more preferably at least 8, amino acids long) which has 10% or greater (more preferably, 50% or greater, and most preferably 75% or greater) sequence identity with a region (preferably within the amino-terminal 34 amino acid region) of hPTH or hPTHrP, and which is capable of blocking the differentiation or the inhibition of proliferation in vitro of cultured human keratinocytes by PTH (1-34) or 1,25(OH)2D3 or PTHrP (1-34); or in vivo in mouse skin by stimulating skin cell proliferation or accelerating hair cycle progression or stimulating hair growth. In a preferred embodiment of this method, the peptide is PTH (7-34), hPTH (5-34) or hPTHrP (5-34). In a related method of the invention, proliferation of mammalian skin cells, e.g., during wound healing, is enhanced by contacting the cell or wound with a liposomal preparation comprising a peptide (preferably at least 3, and more preferably at least 8, amino acids long) which has 10% or greater (more preferably, 50% or greater, and most preferably, 75% or greater) sequence identity with a region (preferably, within the amino-terminal 34 amino acid region) of hPTH or hPTHrP, and which is capable of enhancing wound healing in an in vivo skin punch assay. In preferred embodiments of this method, the peptide is HPTH (1-84), hPTH (1-34), HPTH (7-34), HPTh (5-34), HPTh (5-36), hPTHrP (1-34), or hPTHrP (7-34). These related methods have particular application in the enhancement of wound healing and also have applications in the promotion of skin growth in patients with burns or skin ulcerations as well as in the stimulation of epidermal regrowth in people who have decreased epidermal cell proliferation due to aging.

Hair growth is stimulated by administering to a mammal a liposomal preparation comprising a peptide (preferably at least 3, and more preferably at least 8, amino acids long) which has 10% or greater (more preferably, 50% or greater, and most preferably, 75% or greater) sequence identity with a region (preferably, within the amino-terminal 34 amino acid region) of hPTH or hPTHrP, and which is capable of stimulating hair growth in vitro or in vivo. In preferred embodiments of this method, the peptide is hPTH (7-34), HPTH (5-34) or hPTH (5-36). This method has applications in the promotion of new hair growth or stimulation of the rate of hair growth, e.g., following chemotherapeutic treatment or for treating a form of alopecia, e.g., male or female pattern baldness.

In particular, the invention relates to a method of inhibiting proliferation or enhancing differentiation of a mammalian skin or hair cell, the method comprising topically administering to the mammalian skin or hair cell in need of inhibited proliferation or enhanced differentiation with a proliferation-inhibiting or differentiation-enhancing amount of a peptide or a salt or derivative thereof encapsulated within a liposome, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes; or in vivo in mouse skin by inhibiting skin cell proliferation or hair cycle progression or hair growth; wherein the liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

The invention also relates to a method of inhibiting proliferation or enhancing differentiation of a skin or hair cell of a mammal, comprising administering to the mammal in need thereof a proliferation-inhibiting or differentiation-enhancing amount of a peptide or a salt or derivative thereof and an active vitamin D compound, wherein the peptide is at least 3 amino acids long, has at least 10% sequence identity with the 34 amino acid N-terminal region of HPTH or hPTHrP, and is capable of inhibiting proliferation or enhancing differentiation in vitro of cultured human keratinocytes, or in vivo in mouse skin by inhibiting skin cell proliferation or hair cycle progression or hair cell growth.

The invention also relates to a method of inducing proliferation of a mammalian skin or hair cell, the method comprising topically administering to the mammalian skin or hair cell in need of proliferation with a proliferation-inducing amount of a peptide or a salt or derivative thereof encapsulated within a liposome, wherein the peptide is at least 3 amino acids long, has at least 10% sequence identity with the 34 amino acid N-terminal region of hPTH or hPTHrP, and is capable of blocling the inhibition of proliferation or stimulation of differentiation in vitro of cultured human keratinocytes by PTH (1-34), 1,25(OH)2D3 or PTHrP (1-34), or in vivo in mouse skin by stimulating skin cell proliferation or accelerating hair cycle progression or stimulating hair growth; wherein said liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

The invention also relates to a composition comprising a proliferation-inhibiting or differentiation-enhancing amount of a peptide or a salt or derivative thereof encapsulated within a liposome, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes, or in vivo in mouse skin by inhibiting skin cell proliferation or hair cycle progression or hair growth; wherein said liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

The invention also relates to a composition comprising a proliferation-inducing amount of a peptide or a salt or derivative thereof encapsulated within a liposome, wherein the peptide is at least 3 amino acids long, has at least 10% sequence identity with the 34 amino acid N-terminal region of hPTH or hPTHrP, and is capable of blocking the inhibition of proliferation or stimulation of differentiation in vitro of cultured human keratinocytes by PTH (1-34), 1,25(OH)2D3 or PTHrP (1-34), or in vivo in mouse skin by stimulating skin cell proliferation or accelerating hair cycle progression or stimulating hair growth; wherein said liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

The invention also relates to a composition comprising a proliferation-inhibiting or differentiation-enhancing amount of a peptide or a salt or derivative thereof and an active vitamin D compound, optionally encapsulated within a liposome, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes, or in vivo in mouse skin by inhibiting skin cell proliferation or hair cycle progression or hair growth.

Other features and advantages of the invention will be apparent from the following description of the preferred embodiments thereof, and from the claims.

### BRIEF DESCRIPTION OF THE FIGURES

FIG. 1 depicts a bar graph showing the macroscopic effects of topical PTH (7-34) in Novasome on C57BL/6 mice (7 days).

FIG. 2 depicts a bar graph showing the effects of PTH (7-34) in Novasome on BRDU stained hair follicle cells (7 days).

FIG. 3 depicts a bar graph showing the topical effects of PTH (1-34) in Novasome on BRDU stained hair follicle cells.

FIG. 4 depicts a bar graph showing the effect of 60 days of topical PTH (1-34) on tritiated thymidine incorporation into epidermal DNA in SKH-1 hairless mice.

FIG. 5 depicts a bar graph showing the effect of 60 days of topical PTH (7-34) in Novasome on tritiated thymidine incorporation into epidermal DNA in SKH-1 hairless mice.

### DESCRIPTION OF THE PREFERRED EMBODIMENTS

Synthesis and Selection of Peptides

The peptides used in the methods of the invention are all easily synthesized, using recombinant DNA or solid phase peptide synthesis techniques, and some are available commercially as well, or can be derived from commercially available peptides. For example, there is reproduced below a section of the Bach Chem catalog, listing a number of available human, rat, and bovine analogs and fragments. (The Peninsula Laboratory catalog also lists available fragments.)

| PTHrP - (1-40) | |
|---|---|
| H₂N-Ala-Val-Ser-Glu-His-Gln-Leu-Leu-His-Asp-Lys-Gly-Lys-Ser-Ile-Gln- | (SEQ ID NO:1) |
| Asp-Leu-Arg-Arg-Arg-Phe-Phe-Leu-His-His-Leu-Ile-Ala-Glu-Ile-His-Thr- | |
| Ala-Glu-Ile-Arg-Ala-Thr-Ser-OH | |
| PTH, Bovine (bPTH) (84 amino acids) | |
| H₂N-Ala-Val-Ser-Glu-Ile-Gln-Phe-Met-His-Asn-Leu-Gly-Lys-His-Leu-Ser- | (SEQ ID NO:2) |
| Ser-Met-Glu-Arg-Val-Glu-Trp-Leu-Arg-Lys-Lys-Leu-Gln-Asp-Val-His-Asn- | |
| Phe-Val-Ala-Leu-Gly-Ala-Ser-Ile-Ala-Tyr-Arg-Asp-Gly-Ser-Ser-Gln-Arg- | |
| Pro-Arg-Lys-Lys-Glu-Asp-Asn-Val-Leu-Val-Glu-Ser-His-Gln-Lys-Ser-Leu- | |
| Gly-Glu-Ala-Asp-Lys-Ala-Asp-Val-Asp-Val-Leu-Ile-Lys-Ala-Lys-Pro-Gln- | |
| OH | |
| [Tyr⁶³]-hPTH (63-84) | |
| H₂N-Tyr-Glu-Lys-Ser-Leu-Gly-Glu-Ala-Asp-Lys-Ala-Asp- | (SEQ ID NO:3) |
| Val-Asn-Val-Leu-Thr-Lys-Ala-Lys-Ser-Gln-OH | |
| hPTH (64-84) | |
| H₂N-Glu-Lys-Ser-Leu-Gly-Glu-Ala-Asp-Lys-Ala-Asp-Val-Asn-Val-Leu-Thr- | (SEQ ID NO:4) |
| Lys-Ala-Lys-Ser-Gln-OH | |
| [Tyr⁶⁹]-hPTH (69-84) | |
| H₂N-Tyr-Ala-Asp-Lys-Ala-Asp-Val-Asn-Val-Leu-Thr-Lys-Ala-Lys-Ser-Gln- | (SEQ ID NO:5) |
| OH | |
| hPTH (70-84) | |
| H₂N-Ala-Asp-Lys-Ala-Asp-Val-Asn-Val-Leu-Thr-Lys-Ala-Lys-Ser-Gln-OH | (SEQ ID NO:6) |
| Human Bone Gla Protein (37-49) (BGP 37-49) | |
| H₂N-Gly-Phe-Gln-Glu-Ala-Tyr-Arg-Arg-Phe-Tyr-Gly-Pro-Val-OH | (SEQ ID NO:7) |
| (Poser, J. W. et at, (1980) PNAS 255:8685) | |
| [Tyr³⁶, Phe^{42,46}]-Human Bone Gla Protein (38-49) | |
| H₂N-Tyr-Gln-Glu-Ala-Phe-Arg-Arg-Phe-Phe-Gly-Pro-Val-OH | (SEQ ID NO:8) |
| Human Bone Gla Protein (45-49) | |
| H₂N-Phe-Tyr-Gly-Pro-Val-OH | (SEQ ID NO:9) |
| hPTH (84 amino acids) | |
| H₂N-Ser-Val-Ser-Glu-Ile-Gln-Leu-Met-His-Asn-Leu-Gly-Lys-His-Leu-Asn- | (SEQ ID NO:10) |
| Ser-Met-Glu-Arg-Val-Glu-Trp-Leu-Arg-Lys-Lys-Leu-Gln-Asp-Val-His-Asn- | |
| Phe-Val-Ala-Leu-Gly-Ala-Pro-Leu-Ala-Pro-Arg-Asp-Ala-Gly-Ser-Gln-Arg- | |
| Pro-Arg-Lys-Lys-Glu-Asp-Asn-Val-Leu-Val-Glu-Ser-His-Glu-Lys-Ser-Leu- | |
| Gly-Glu-Ala-Asp-Lys-Ala-Asp-Val-Asn-Val-Leu-Thr-Lys-Ala-Lys-Ser-Gln- | |
| OH | |
| (Kimura, T. et al, (1983) BBRC 114493; Fairwell, T. et | |
| al, (1983) Biochemistry 222691) | |
| Rat PTH (rPTH) (84 amino acids) | |
| H₂N-Ala-Val-Ser-Glu-Ile-Gln-Leu-Met-His-Asn-Leu-Gly-Lys-His-Leu-Ala- | (SEQ ID NO:11) |
| Ser-Val-Glu-Arg-Met-Gln-Trp-Leu-Arg-Lys-Lys-Leu-Gln-Asp-Val-His-Asn- | |
| Phe-Val-Ser-Leu-Gly-Val-Gln-Met-Ala-Ala-Arg-Glu-Gly-Ser-Tyr-Gln-Arg- | |
| Pro-Thr-Lys-Lys-Glu-Asp-Asn-Val-Leu-Val-Asp-Gly-Asn-Ser-Lys-Ser-Leu- | |
| Gly-Glu-Gly-Asp-Lys-Ala-Asp-Val-Asp-Val-Leu-Val-Lys-Ala-Lys-Ser-Gln- | |
| OH | |
| (Heinrich, G. et al, (1984) J. Biol. Chem. 2593320) | |
| bPTH (1-34) | |
| H₂N-Ala-Val-Ser-Glu-Ile-Gln-Phe-Met-His-Asn-Leu-Gly-Lys-His-Leu-Ser- | (SEQ ID NO:12) |
| Ser-Met-Glu-Arg-Val-Glu-Trp-Leu-Arg-Lys-Lys-Leu-Gln-Asp-Val-His-Asn- | |
| Phe-OH | |
| (Tregear, G. W. et al, (1977) Biochemistry 162817) | |
| hPTH (1-34) | |
| H₂N-Ser-Val-Ser-Glu-Ile-Gln-Leu-Met-His-Asn-Leu-Gly-Lys-His-Leu-Asn- | (SEQ ID NO:13) |
| Ser-Met-Glu-Arg-Val-Glu-Trp-Leu-Arg-Lys-Lys-Leu-Gln-Asp-Val-His-Asn- | |
| Phe-OH | |
| (Takel, T. et al, (1979) Peptide Chemistry) | |
| [Nle^{8,18}, Tyr³⁴]-bPTH (1-34), Amide | |
| H₂N-Ala-Val-Ser-Glu-Ile-Gln-Phe-Nle-His-Asn-Leu-Gly-Lys-His-Leu-Ser- | (SEQ ID NO:14) |
| Ser-Nle-Glu-Arg-Val-Glu-Trp-Leu-Arg-Lys-Lys-Leu-Gln-Asp-Val-His-Asn- | |
| Tyr-NH₂ | |
| (Colrora, M. D. et al, (1981) J. Biol. Chem. 256:10.555; Rosenblatt, | |
| M. et al, (1977) Endocr. Res. Comm. 4:115; Rosenblatt, M. et al, | |
| (1976) J. Biol. Chem. 251:159) | |
| [Nle^{8,18}, Tyr³⁴] hPTH (1-34) | |
| H₂N-Ser-Val-Ser-Glu-Ile-Gln-Leu-Nle-His-Asn-Leu-Gly-Lys-His-Leu-Asn- | (SEQ ID NO:15) |
| Ser-Nle-Glu-Arg-Val-Glu-Trp-Leu-Arg-Lys-Lys-Leu-Gln-Asp-Val-His-Asn- | |
| Tyr-OH | |
| [Nle^{8,21}, Tyr³⁴]-rPTH (1-34), Amide | |
| H₂N-Ala-Val-Ser-Glu-Ile-Gln-Leu-Nle-His-Asn-Leu-Gly-Lys-His-Leu-Ala- | (SEQ ID NO:16) |
| Ser-Val-Glu-Arg-Nle-Gln-Trp-Leu-Arg-Lys-Lys-Leu-Gln-Asp-Val-His-Asn- | |
| Tyr-NH₂ | |
| [Tyr¹]-hPTH (1-34) | |
| H₂N-Tyr-Val-Ser-Glu-Ile-Gln-Leu-Met-His-Asn-Leu-Gly-Lys-His-Leu-Asn- | (SEQ ID NO:17) |
| Ser-Met-Glu-Arg-Val-Glu-Trp-Leu-Arg-Lys-Lys-Leu-Gln-Asp-Val-His-Asn- | |
| Phe-OH | |
| hPTH (1-38) | |
| H₂N-Ser-Val-Ser-Glu-Ile-Gln-Leu-Met-His-Asn-Leu-Gly-Lys-His-Leu-Asn- | (SEQ ID NO:18) |
| Ser-Met-Glu-Arg-Val-Glu-Trp-Leu-Arg-Lys-Lys-Leu-Gln-Asp-Val-His-Asn- | |
| Phe-Val-Ala-Leu-Gly-OH | |
| (Heech, R. D. et al, (1984) Horm. Metab. Res. 16:556) | |
| hPTh (1-44) | |
| H₂N-Ser-Val-Ser-Glu-Ile-Gln-Leu-Met-His-Asn-Leu-Gly-Lys-His-Leu-Asn- | (SEQ ID NO:19) |
| Ser-Met-Glu-Arg-Val-Glu-Trp-Leu-Arg-Lys-Lys-Leu-Gln-Asp-Val-His-Asn- | |
| Phe-Val-Ala-Leu-Gly-Ala-Pro-Leu-Ala-Pro-Arg-OH | |
| (Kimura T. et al, (1981) Biopolymers 20:1823) | |
| bPTH (3-34) | |
| H₂N-Ser-Glu-Ile-Gln-Phe-Met-His-Asn-Leu-Gly-Lys-His-Leu-Ser-Ser-Met- | (SEQ ID NO:20) |
| Glu-Arg-Val-Glu-Trp-Leu-Arg-Lys-Lys-Leu-Gln-Asp-Val-His-Asn-Phe-OH | |
| (Lowrik, C. et al, (1985) Cell Calcium 6:311) | |
| [Nle^{8,18}, Tyr³⁴]-bPTH (3-34), Amide | |
| H₂N-Ser-Glu-Ile-Gln-Phe-Nle-His-Asn-Leu-Gly-Lys-His-Leu-Ser-Ser-Nle- | (SEQ ID NO:21) |
| Glu-Arg-Val-Glu-Trp-Leu-Arg-Lys-Lys-Leu-Gln-Asp-Val-His-Asn-Tyr-NH₂ | |
| (Rosenblatt, M. et al, (1977) J. Biol. Chem. 252:5647) | |
| [Nle^{8,18}, Tyr³⁴]-bPTH (7-34), Amide | |
| H₂N-Phe-Nle-His-Asn-Leu-Gly-Lys-His-Leu-Ser-Ser-Nle-Glu-Arg-Val-Glu- | (SEQ ID NO:22) |
| Trp-Leu-Arg-Lys-Lys-Leu-Gln-Asp-Val-His-Asn-Tyr-NH₂ | |
| [Tyr³⁴]-bPTH (7-34), Amide | |
| H₂N-Phe-Met-His-Asn-Leu-Gly-Lys-His-Leu-Ser-Ser-Met-Glu-Arg-Val-Glu- | (SEQ ID NO:23) |
| Trp-Leu-Arg-Lys-Lys-Leu-Gln-Asp-Val-His-Asn-Tyr-NH₂ | |
| hPTH (13-34) | |
| H₂N-Lys-His-Leu-Asn-Ser-Met-Glu-Arg-Val-Glu-Trp-Leu-Arg-Lys-Lys-Leu- | (SEQ ID NO:24) |
| Gln-Asp-Val-His-Asn-Phe-OH | |
| [Tyr²⁷]-hPTH (27-48) | |
| H₂N-Tyr-Leu-Gln-Asp-Val-His-Asn-Phe-Val-Ala-Leu-Gly-Ala-Pro-Leu-Ala- | (SEQ ID NO:25) |
| Pro-Arg-Asp-Ala-Gly-Ser-OH | |
| hPTH (28-48) | |
| H₂N-Leu-Gln-Asp-Val-His-Asn-Phe-Val-Ala-Leu-Gly-Ala-Pro-Leu-Ala-Pro- | (SEQ ID NO:26) |
| Arg-Asp-Ala-Gly-Ser-OH | |
| Rosenblatt, M. et al, (1977) Biochemistry 16:2811) | |
| hPTH (53-84) | |
| H₂N-Lys-Lys-Glu-Asp-Asn-Val-Leu-Val-Glu-Ser-His-Glu-Lys-Ser-Leu-Gly- | (SEQ ID NO:27) |
| Glu-Ala-Asp-Lys-Ala-Asp-Val-Asn-Val-Leu-Thr-Lys-Ala-Lys-Ser-Gln-OH | |
| (Rosenblatt, M. et al, (1978) Endocrinology 103:976) | |

In addition, the peptides and peptide derivatives disclosed in the following documents can also be used: U.S. Pat. Nos. 4,086,196, 4,423,037, 4,771,124, 4,833,125, 4,968,669, 5,001,223, 5,087,562, 5,093,233, 5,116,952, 5,149,779, 5,171,670, 5,229,489, 5,317,010, 5,382,658, 5,393,869, 5,434,246, 5,527,772, 5,589,452, 5,807,823, 5,821,255, 5,840,690, 5,977,070, 6,025,467, 6,051,868, and 6,066,618; WO094/02510, WO00/23594, and WO00/31137; and EP 477,885.

When selecting a candidate peptide for a method of this invention, a preferred first step is to choose a peptide which includes a fragment which has at least 10%, and more preferably 50% or greater, sequence identity with an 8 or greater amino acid long fragment within the amino terminal 34 amino acid region of hPTH or hPTHrP. The term “sequence identity” refers to a measure of the identity of nucleotide sequences or amino acid sequences. In general, the sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(i):387 (1984)), BLASTP, BLASTN, FASTA (Atschul, S.F., et al., J Molec Biol 215:403 (1990)).

Therefore, as used herein, the term “identity” represents a comparison between a test and reference polypeptide. More specifically, reference test polypeptide is defined as any polypeptide that is 10% or more identical to a reference polypeptide. As used herein, the term at least 10% identical to refers to percent identities from 10 to 99.99 relative to the reference polypeptides. Identity at a level of 10% or more is indicative of the fact that, assuming for exemplification purposes a test and reference polynucleotide length of 100 amino acids, that no more than 90% (i.e., 90 out of 100) amino acids in the test polypeptides differ from that of the reference polypeptides. Such differences may be represented as point mutations randomly distributed over the entire length of the amino acid sequence of the invention or they may be clustered in one or more locations of varying length up to the maximum allowable amino acid difference. Differences are defined as amino acid substitutions, or deletions.

Because of the high degree of homology among human PTH and PTH of other species, non-human as well as human fragments or analogs can be used. Further, the fragment can be modified in any of a variety of standard chemical ways, e.g., the carboxy-terminal amino acid residue can be made into a terminal amide group; the amino-terminal residue can be modified with groups to, e.g., enhance lipophilicity; the peptide can be chemically glycosylated to increase solubility or in vivo half-life; and D-amino acids can be substituted for L-isomers in the peptide.

Candidate peptides may be tested for suitability as inhibitors of cell proliferation and enhancers of differentiation using cultured human keratinocytes, as described in U.S. Pat. Nos. 5,527,772, 5,840,690 and 6,066,618. Briefly, those peptides which inhibit proliferation and induce differentiation in cultured keratinocytes are those potentially useful as therapeutic agents in treating disorders, e.g., psoriasis and cancer, where suppression of cell proliferation is desired. Candidate peptides may be tested for suitability as enhancers of cell proliferation using cultured human keratinocytes or in vivo mouse model. Those peptides which block the effect of agonist peptides or 1,25(OH)2D3 on cultured keratinocyte proliferation are those potentially useful as therapeutic agents in treating disorders, e.g., wounds, bums, or skin ulcerations, where maintenance or stimulating of cell proliferation is desired.

Candidate peptides may be tested for their ability to enhance wound healing by carrying out a skin punch biopsy test, as described in U.S. Pat. Nos. 5,527,772, 5,840,690 and 6,066,618.

Candidate peptides may be tested for suitability as stimulators of hair growth using an in vitro hair growth assay, as described in U.S. Pat. Nos. 5,527,772, 5,840,690 and 6,066,618. Those peptides which stimulate hair growth in vitro are those potentially useful for the stimulation of hair growth in vivo, e.g., for the stimulation or maintenance of hair growth during or following chemotherapy or to treat a form of alopecia, e.g., male pattern baldness.

Alternatively, in vivo assays may be carried out as described herein and in Schilli, M. B. et al., J. Invest. Dermatol. 108:928-932 (1997); Holick, M. F., et al., Proc. Natl. Acad Sci. 91:8014-8016 (1994); Paus, R and Cotsarelis, G., N. Engl. J. Med 341: 491-497 (1999); and Paus, R, et al. Laboratory Invest. 60: 365-369 (1989).

Peptides which block antiproliferative compounds can also be useful in conjunction with chemotherapeutic agents in the treatment of skin cancer; many chemotherapeutic agents are effective only against dividing cells, and the blocking peptides can have the effect of inducing division of otherwise dormant cells, rendering them vulnerable to the chemotherapy. Blocking peptides can also be useful in promoting growth of new cells, e.g., skin cells, in topical skin creams. Differentiation-inducing peptides can be used as immunostimulants, by inducing maturation of monocytes and lymphocytes bearing PTH receptors, while blocking peptides can be used to inhibit lymphocyte maturation, and thus can be used to treat conditions, e.g., autoimmune diseases such as juvenile diabetes, rheumatoid arthritis, and allograft rejection, where mature lymphocytes are a causative agent.

The peptides are administered in therapeutically effective amounts to mammals in need of them. The peptides may be administered as part of liposomal preparations described in U.S. Pat. No. 5,260,065. Such liposomes comprise at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

The preferred primary lipids are C₁₂-C₁₈ fatty alcohols, glyceryl mono- and distearate, glyceryl dilaurate, and glycol stearate. While any of the secondary lipids could be used with any of the primary lipids, preferred combinations include polyoxyethylene 10-20 acyl alcohols or quaternary dirnethyldiacyl amines as the secondary lipids to be used in conjunction with the fatty alcohols. Matching chain lengths in terms of carbon content and ursaturations is an important factor to consider for selection of the secondary lipid. These same acyl alcohols and dimethyldiacyl (specifically distearyl) amines are also useful with the glycol stearate, glyceryl monostearate, glyceryl distearate and the glyceryl dilaurate. However, the glyceryl distearate and glyceryl dilaurate may also use sodium laurate sarcosinates, as well as other matching sarcosinate salts (all being water soluble), or lauryl sarcosinates as secondary lipids.

In certain instances, primarily the stearate derivatives, a sterol such as cholesterol is a particularly useful additive. The addition of cholesterol appears to make the vesicles population more uniform in terms of size and shape. Even cholesterol is not sufficient, in itself, to allow vesicle formation. This is contrast to the materials described in U.S. Pat. No. 4,917,951 which only require cholesterol to make vesicles. In certain circumstances, cholesterol will allow these materials which will not otherwise form a lamellar phase to form a lamellar phase but they cannot be formed into vesicles without the addition of the secondary lipid. In fact, some of the most preferred secondary lipids, e.g., dimethyldistearyl amine, water soluble polyoxyethylene acyl alcohols, and acyl sarcosinate salts, will not form vesicles or lamellar phases either.

According to Example 1 of U.S. Pat. No. 5,260,065, a variety of materials may be blended in order to make vesicles. Table 1 shows the composition, water uptake level, and oil uptake under hot and cold loading techniques of five different compositions. According to U.S. Pat. No. 5,260,065, none of the primary lipids used, e.g., glyceryl dilaurate (GDL), glyceryldistearate (GDS), cetyl alcohol (CA), stearyl alcohol (SA), or glycol stearate (GS) will form vesicles or lamellar phase on their own.

TABLE 1

| | Water Uptake | Oil Uptake (ml/ml) | |
|---|---|---|---|
| Composition | (ml/ml) | Hot | Cold |
| GDL/C16Q/Chol | 13.5 | ≧7.2 | ≧2.7 |
| (1.0/0.05/0.05) | | | |
| GDS/POE10SA/Chol | 12.5 | ≧6.9 | ≧6.5 |
| (1.0/0.5/0.25) | | | |
| CA/POE10CA/Chol | 9.5 | ≧4.2 | ≧4.2 |
| (1.0/0.2/0.1) | | | |
| SA/C18Q/Chol | 13.5 | ≧6.5 | ≧6.5 |
| (1.0/0.2/0.1) | | | |
| GS/POE10SA/Chol | 13.5 | ≧6.5 | ≧6.5 |
| (1.0/0.2/0.1) | | | |

The first compound shown in Table 1 is a blend of glyceryl dilaurate, dimethyldicetyl quaternary amine (C16Q), and cholesterol (Chol) in a 1.0:0.05:0.05 molar ratio. According to U.S. Pat. No. 5,260,065, the water uptake is 13.5 ml/ml of lipid and the hot load and cold loading values were ≧7.2 and ≧2.7 ml of oil/ml of lipid, respectively. According to U.S. Pat. No. 5,260,065, the vesicles were made by blending the two lipids and the cholesterol at 70°-75° C. with the aqueous phase at 65° C. According to U.S. Pat. No. 5,260,065, the lipid phase was placed in one syringe, the aqueous phase was placed in another syringe, and the two syringes were connected by a stopcock According to U.S. Pat. No. 5,260,065, the material was shear mixed by blending from one syringe to another through the stopcock forming vesicles in less than two minutes. According to U.S. Pat. No. 5,260,065, for the cold loading technique, the preformed vesicles were mixed with 20% and 50% V/V mineral oil (Drakeol 19) using the same syringe technique to load the oil. According to U.S. Pat. No. 5,260,065, for the hot loading technique, the oil was heated to 70°-75° C., blended with the lipophilic phase prior to hydration by the aqueous phase, and then the combined lipophilic/water immiscible oily phase was hydrated by the aqueous phase. According to U.S. Pat. No. 5,260,065, either hot loading or cold loading techniques may be used for a mineral oil but with a highly volatile oil which would not survive the 70°-75° C. heating, the cold loading technique, which can be carried out a ambient temperature, is preferred. According to U.S. Pat. No. 5,260,065, the second compounded tested was a blend of glyceryl distearate, Polyoxyethylene 10 stearyl alcohol (POE10SA), and cholesterol in a 1.0:0.5:0.25 molar ratio. This blended material had a water uptake of 12.5 ml/ml lipid and the oil uptake for either hot and cold loading was >6.5 ml/ml using the same techniques previously described. According to U.S. Pat. No. 5,260,065, the third material tested was a blend of cetyl alcohol, polyoxyethylene 10 cetyl alcohol (POE10CA), and cholesterol in a 1:0.2:0.1 molar ratio. Water uptake was 9.5 ml/ml and both hot and cold oil uptake was >4.2 ml/ml lipid.

According to U.S. Pat. No. 5,260,065, the fourth combination tested was a blend of stearyl alcohol, dimethyldistearyl quaternary amine (C18Q), and cholesterol on a 1:0.2:0.1 ratio. Water uptake was 13.5 ml/ml and oil uptake on both a hot and cold basis was >6.5 ml/ml lipid.

According to U.S. Pat. No. 5,260,065, the fifth compound tested was a blend of glycol stearate, polyoxyethylene 10 stearyl alcohol, and cholesterol in a 1:0.2:0.1 ratio. Again, the water uptake was approximately 13.5 ml/ml and the oil uptake was >6.5 ml/ml under both hot and cold loading techniques. According to Example 2 of U.S. Pat. No. 5,260,065, retinoic acid, a water insoluble material in a water immiscible carrier, was used in lieu of the mineral oil of Example 1 in the amorphous central cavity of the paucilamellar lipid vesicles. Retinoic acid has a substantial number of dermatological uses including, potentially, the reduction of facial wrinkles.

TABLE 2

| | A | | B | |
|---|---|---|---|---|
| Cetyl Alcohol | 4.7 | g | | |
| Glycol Stearate | | | 11.5 | g |
| POE10 Cetyl Alcohol | 2.35 | g. | | |
| POE10 Stearyl Alcohol | | | 2.3 | g |
| Cholesterol | 1.2 | g | 1.15 | g |
| Petrolatum | 10.9 | g | | |
| Paraffin Wax | 11.6 | g | | |
| Soybean Oil | | | 21.8 | g |
| Retinoic Acid | 0.25 | g | 0.25 | g |
| Deionized Water | 69 | g | 63 | g |

According to U.S. Pat. No. 5,260,065, Table 2 shows the formulas for two different retinoic acid formulations, one using a cetyl alcohol/polyoxyethylene 10 cetyl alcohol blend and the other using a glycol stearate/polyoxyethylene 10 stearyl alcohol blend as the vesicles formers. According to U.S. Pat. No. 5,260,065, both formulas include cholesterol while one uses a mixture petrolatum and paraffin wax as a carrier for the retinoic acid while the other uses a soybean oil carrier. According to U.S. Pat. No. 5,260,065, in both cases, the retinoic acid was dissolved in the carrier at 65°-75° C. According to U.S. Pat. No. 5,260,065, the lipids and the cholesterol were then heated and blended to homogeneity and the retinoic acid mixture was added and blended therein. According to U.S. Pat. No. 5,260,065, an aqueous phase consisting of the deionized water was then heated to approximately 65° C. and the resulting phases were shear mixed to form the vesicles. According to U.S. Pat. No. 5,260,065, while the syringe method described in Example 1 could be used, a NovaMix™ vesicle forming machine manufactured by Micro Vesicular Systems, Inc., Nashua, N.H. was used. This machine, which is described in more detail in U.S. Pat. No. 4,895,452, has a substantially cylindrical central chamber with an axial outflow tube and tangentially located inflow tubes. According to U.S. Pat. No. 5,260,065, the phases are injected into the central chamber, under pressure sufficient to form turbulent flow and shear mixing, rapid vesicle formation occurs, and the vesicles are removed through the outflow tube.

Alternatively, the apparatus described in U.S. Pat. No. 5,013,497 may be used to prepare the liposomes.

According to Example 3 of U.S. Pat. No. 5,260,065, two different formulations for encapsulating anthralin, an antipsoriatic, were tested. Table 3 lists the ingredients used in these formulations. According to the present invention, a peptide agonist or antagonist may be substituted for anthralin.

TABLE 3

| | C | | D | |
|---|---|---|---|---|
| Glyceryl Distearate | 9.4 | g | | |
| Cetyl Alcohol | | | 6.85 | g |
| Dimethyl Distearyl Ammonium Chloride | 0.3 | g | | |
| POE10 Cetyl Alcohol | | | 1.35 | g |
| Sodium Lauryl Sarcosinate | 1.4 | g | | |
| Cholesterol | 1.0 | g | 0.7 | g |
| Petrolatum | 15.7 | g | 17.3 | g |
| Paraffin Wax | 16.8 | g | 18.5 | g |
| Anthralin | 0.5 | g | 0.5 | g |
| Deionized Water | 54.9 | g | 54.8 | g |

According to U.S. Pat. No. 5,260,065, in formulation C, the petrolatum and paraffin are melted together and the anthralin is dissolved into the carrier mixture. According to U.S. Pat. No. 5,260,065, this also the case of formulation D. According to U.S. Pat. No. 5,260,065, this petrolatum/paraffin wax mixture appears to be particularly advantageous in that micro-crystals form rather than the macroscopic crystals which normally appear when anthralin cools. According to U.S. Pat. No. 5,260,065, in formulation C, however, the glyceryl distearate, cholesterol and dimethyldistearyl ammonium chloride are blended together at approximately 75° C. until clear and the anthralin solution (forming a water immiscible phase) is then mixed therein. According to U.S. Pat. No. 5,260,065, the aqueous phase is formed by heating the deionized water to approximately 65° C. and dissolving the secondary lipid, the sodium lauryl sarcosinate, therein. According to U.S. Pat. No. 5,260,065, the aqueous phase and the lipid phase are then shear mixed, using a NovaMix™ machine as described in Example 2, to form vesicles. According to U.S. Pat. No. 5,260,065, in contrast, in formulation D, the cetyl alcohol, polyoxyethylene 10 cetyl alcohol and the cholesterol are blended together at an elevated temperature, the anthralin solution is mixed in, and the aqueous which consists merely of the deionized water is shear mixed using the NovaMix™ machine to form the vesicles. According to U.S. Pat. No. 5,260,065, the difference in the procedure is that the non-ionic lipids of formulation D cannot be carried in the aqueous solution as is the ionic sodium lauryl sarcosinate of formulation C. According to U.S. Pat. No. 5,260,065, either formulation forms acceptable anthralin carrying vesicles.

According to Example 4 of U.S. Pat. No. 5,260,065, three different materials, Vitamin E acetate, levamisole base, and a butter flavor oil were carried in the central cavity of vesicles of the invention. Table 4 shows the formulas for these vesicles. According to the present invention, a peptide agonist or antagonist may be substituted for vitamin E acetate.

TABLE 4

| | E | | F | | G | |
|---|---|---|---|---|---|---|
| Glyceryl Distearate | 11.2 | g | | | 4.35 | g |
| Glycol Stearate | | | 7.5 | g | | |
| POE10 Stearyl Alcohol | 5.6 | g | 1.5 | g | 2.2 | g |
| Cholesterol | 2.8 | g | 0.75 | g | 1.1 | g |
| Soybean Oil | | | 8.5 | g | | |
| Vitamin E | 2.2 | g | | | | |
| Levamisole Base | | | 4.63 | g | | |
| Butter Flavor Oil | | | | | 20.0 | g |
| Deionized Water | 78.2 | g | 74.12 | g | 72.35 | g |

According to U.S. Pat. No. 5,260,065, formulation E uses glyceryl distearate, polyoxyethylene 10 stearyl alcohol, and cholesterol as the lipophilic phase which are blended at 70° C. to obtain a clear, homogeneous solution. According to U.S. Pat. No. 5,260,065, the Vitamin E acetate was dissolved therein and the mixture was hydrated with 65° C. water using the NovaMix™ machine as described in Example 2.

According to U.S. Pat. No. 5,260,065, formulation F used a levamisole base (a sheep dip) in soybean oil at 75° C. to form the water immiscible phase. According to U.S. Pat. No. 5,260,065, the glycol stearate, polyoxyethylene stearyl alcohol and cholesterol were heated together at 75° C. to obtain a clear, homogeneous solution and the levamisole/soybean oil mixture was blended therewith. According to U.S. Pat. No. 5,260,065, the deionized water was heated to approximately 65° C. and used as a hydrating solution for the lipids, again using the previously described NovaMix™ machine.

According to U.S. Pat. No. 5,260,065, in formulation G, the lipids and cholesterol were melted together at 750° C. and the butter oil dissolved therein. According to U.S. Pat. No. 5,260,065, again, the deionized water was heated to approximately 65° C. and used as a hydrating solution in a NovaMix™ machine.

According to Example 5 of U.S. Pat. No. 5,260,065, three different formulations for vesicles using retinoic acid, with both cationic and anionic vesicles may be used. Table 5 lists the formulations for each vesicle. According to the present invention, a peptide agonist or antagonist may be substituted for retinoic acid.

TABLE 5

| | H | | I | | J | |
|---|---|---|---|---|---|---|
| Glyceryl Distearate | 9.4 | g | | | | |
| Glycol Stearate | | | 13.2 | g | 13.2 | g |
| Dimethyl Distearyl | 0.3 | g | | | | |
| Ammonium Chloride | | | | | | |
| Dimethyl Dicetyl | | | 0.6 | g | | |
| Ammonium Chloride | | | | | | |
| Sodium Oleate | | | | | 1.0 | g |
| Petrolatum | 15.7 | g | | | | |
| Paraffin Wax | 16.8 | g | | | | |
| Soybean Oil | | | 22.0 | g | 22.0 | g |
| Retinoic Acid | 0.25 | g | 0.25 | g | 0.25 | g |
| Deionized Water | 56.55 | g | 62.75 | g | 63.35 | g |

According to U.S. Pat. No. 5,260,065, formulation H uses the paraffin wax/petrolatum carrier for the retinoic acid, with the retinoic acid being dissolved in the carrier at approximately 65°-75° C. According to U.S. Pat. No. 5,260,065, the lipophilic phase is formed of glyceryl distearate, cholesterol, and the dimethyl distearyl ammonium chloride. According to U.S. Pat. No. 5,260,065, the carrier containing the retinoic acid is blended into the lipophilic phase and is hydrated with the deionized water using the NovaMix™ machine as described in Example 2.

According to U.S. Pat. No. 5,260,065, formulations I and J use the soybean oil carrier and the same materials except for the secondary lipid. According to U.S. Pat. No. 5,260,065, in formulation I, the secondary lipid, which forms part of the initial lipophilic phase, is dimethyl dicetyl ammonium chloride while in formulation J, the secondary lipid, which is incorporated into the aqueous phase, is sodium oleate. According to U.S. Pat. No. 5,260,065, in either case, the retinoic acid is dissolved in the soybean oil at elevated temperatures, the soybean oil is blended into the lipophilic phase, and the combined phase is then hydrated using the aqueous phase. According to U.S. Pat. No. 5,260,065, formulation J forms anionic vesicles while formulation I forms cationic vesicles. However, according to U.S. Pat. No. 5,260,065, both are effective in encapsulating the retinoic acid.

The liposome encapsulted peptides can be admixed with a pharmacologically inert topical carrier such as one comprising a gel, an ointment or a cream, including such carriers as water, glycerol, alcohol, propylene glycol, fatty alcohol, triglycerides, fatty acid ester or mineral oils. Other possible carriers are liquid petrolatum, isopropylpalmitate, polyethylene glycol ethanol 95%, polyoxyethylene monolaurate 5% in water, sodium lauryl sulfate 5% in water, and the like. Materials such as antioxidants, humectants, viscosity stabilizers and the like may be added, if necessary.

The peptides can be provided in the form of pharmaceutically acceptable salts. Examples of preferred salts are those of therapeutically acceptable organic acids, e.g., acetic, lactic, maleic, citric, malic, ascorbic, succinic, benzoic, salicylic, methanesulfonic, toluenesulfonic, or parmoic acid, as well as polymeric acids such as tannic acid or carboxymethyl cellulose, and salts with inorganic acids such as hydrohalic acids, e.g, hydrochloric acid, sulfuric acid, or phsophoric acid.

Dosage will be dependent upon the age, health, and weight of the recipient; kind of concurrent treatment, if any; frequency of treatment; and the nature of the effect desired. Generally, daily dosage will be from about 0.0001 micrograms/kg to 100 micrograms/kg, preferably 0.001 to 10.0 micrograms/kg. The topical dosage will be from about 0.01 micrograms/cm² to 100 Micrograms/cm², preferably 0.1 to 10 micrograms/cm². The liposomal formulations may be applied by one or more applications per day.

The invention also relates to compositions comprising a proliferation-inhibiting or differentiation-enhancing amount of a peptide or a salt or derivative thereof, an active vitamin D compound and a pharmaceutical carrier, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes, or in vivo in mouse skin by inhibiting skin cell proliferation or hair cycle progression or hair growth. A large number of active vitamin D compounds are known which can be used in the practice of the present invention. See U.S. Pat. Nos. 5,457,217, 5,414,098, 5,384,313, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791. A preferred active vitamin D compound is calcipotriene. In this embodiment, any conventional liposome may be used including the liposomes described in U.S. Pat. Nos. 4,235,871, 4,241,046, 4,247,411, 4,356,167, 4,377,567, 4,544,545, 4,551,288, 4,610,868, 4,731,210, 4,744,989, 4,772,471, 4,897,308, 4,917,951, 5,021,200, 5,032,457, and 5,260,065.

The invention relates as well to a method of inhibiting proliferation or enhancing differentiation of a skin or hair cell of a mammal, comprising administring to the mammal in need thereof a proliferation-inhibiting or differentiation-enhancing amount of a peptide or a salt or derivative thereof and an active vitamin D compound, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes, or in vivo in mouse skin by inhibiting skin cell proliferation or hair cycle progression or hair cell growth. Surprisingly, it has been discovered by recalcitrant psoriasis responds to the administration of the peptide and active vitamin D compound. In this embodiment, the peptide and the active vitamin D compound may be administered as part of single or separate pharmaceutical compositions. Either one or both of the peptide and active vitamin D compound may be administered topically or parenterally. In a preferred embodiment, the peptide is administered first followed by the active vitamin D compound.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### Process for the Production of Liposomes

PTH (1-34) and PTH (7-34) was formulated in Novasome A (obtained from IGI, Inc., Buena, N.J.). The formulation included dissolving the PTH analog in doubly distilled water at a concentration of 1 mg/100 μl. This solution was mixed with 3 ml of Novasome A.

### EXAMPLE 2

### Effect of Liposome Encapsulated Peptides on Skin and Hair Growth in Mice

Evaluation of the Effect of Topically Administered PTH (1-34) and PTH (7-34) Formulated in Novasome A on Skin and Hair Growth

C57 BL/6 were purchased from Jackson Labs and were in their telogen state of hair development. The animals were depilated as previously described (1). Groups of six animals received daily a topical application of either 10 μg of PTH (1-34) in 30 μl of Novasome or their depilatated backs, 10 μg of PTH (7-34)/30 μl of Novasome, or 30 μl of Novasome. The animals were dosed daily for seven days. Macroscopic evaluation and tritiated thymidine and bromouridine analysis was made on day seven as previously described (1, 2, 3, 4).

Results

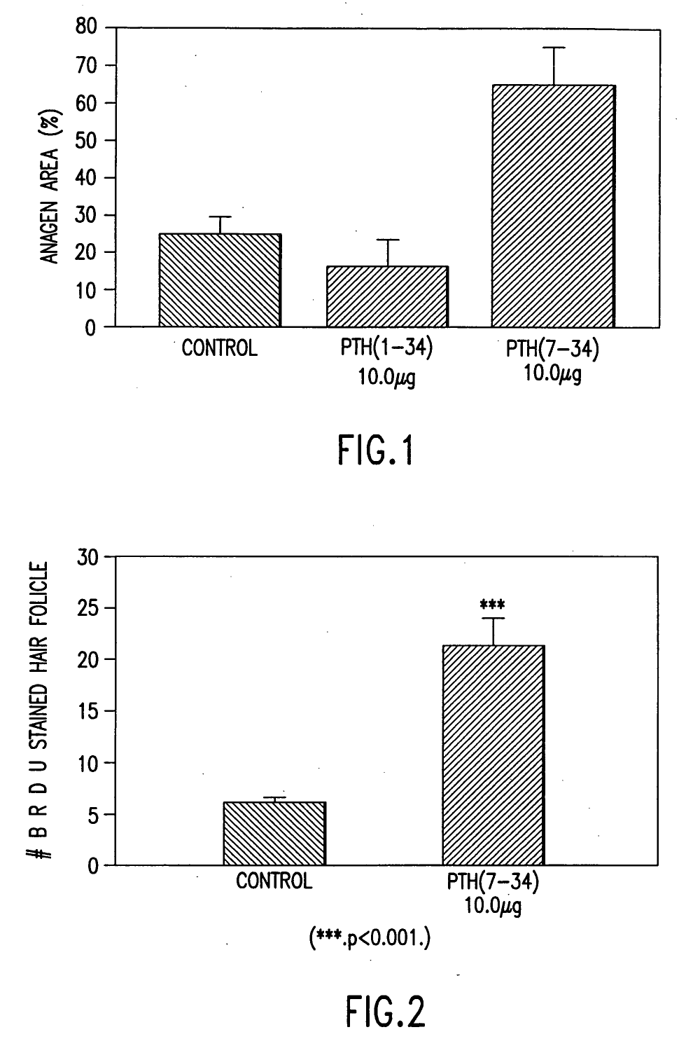

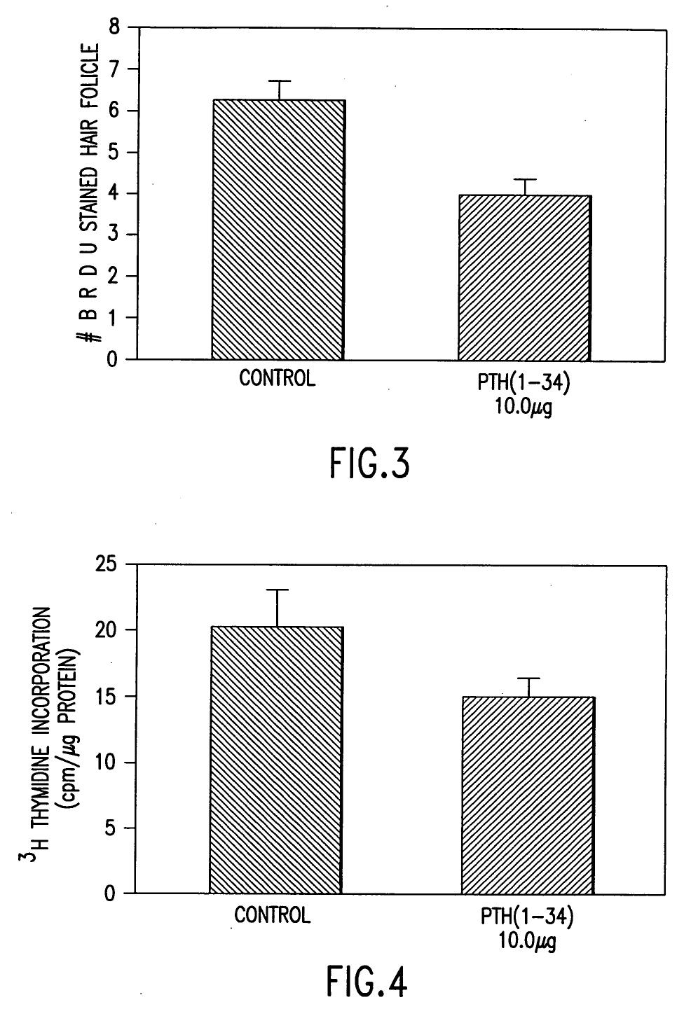

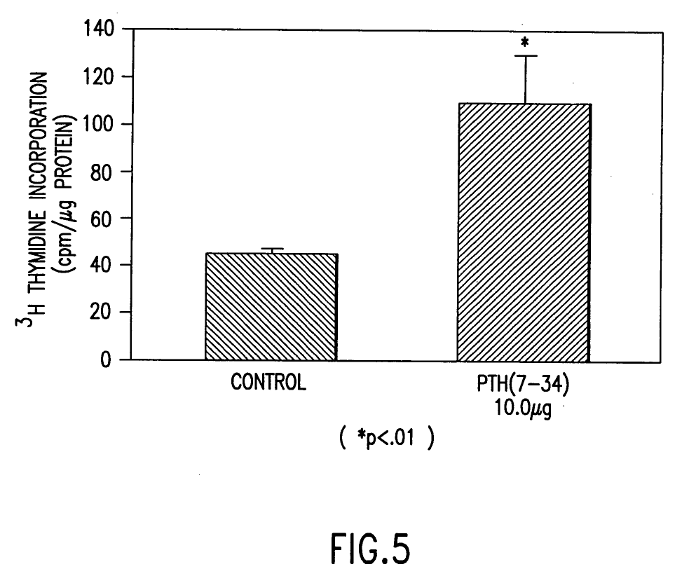

Macroscopic evaluation of the effect of PTH (7-34) on C57 BL/6 mice demonstrated an increase in the progression of the hair follicles into their proliferative anagen state (FIG. 1). There was a 155% increase in the anagen area in the group of animals receiving 10 μg of PTH (7-34) formulated in Novasome A. An evaluation of BRDU staining of the hair follicles revealed that there was an increase in BRDU staining of the hair follicle which was an indication of increased proliferation of the hair follicle (FIG. 2). Animals that received the PTH (1-34) topically showed a decrease in hair growth progression (FIG. 1). An evaluation of the BRDU staining in hair follicles from the mice that received topically 10 μg of PTH (1-34) demonstrated a decrease (FIG. 3).

Conclusion

These results demonstrate that the topical application of PTH (7-34) is able to accelerate the hair cycle and stimulate the hair follicles to proliferate. The animals that received topically PTH (1-34) revealed a decrease in hair growth progression and a decrease in the proliferation of the hair follicles.

Next, the effect of topically applying PTH (1-34) or PTH (7-34) formulated in Novasome A on SKH-1 hairless mice for 60 days was determined.

SKH-1 hairless mice received topically daily for 60 days either 10 μg of PTH (1-34) formulated in Novasome A, PTH (7-34) formulated in Novasome A, or Novasome A without any PTH analog to serve as the control group. The animals received ³H-tritiated thymidine for the evaluation of epidermal proliferation as previously described.

Results

As can be seen in FIG. 4, the animals that received topically PTH (1-34) in Novasome A showed a decrease of 25% in DNA synthesis (proliferation) compared to the control group.

Evaluation of ³H-thymidine in the epidermis of the mice that received topically PTH (7-34) showed a more than two fold enhancement in DNA synthesis (proliferation) compared to the control group 10 μg of PTH (7-34) formulated in Novasome, respectively.

### EXAMPLE 3

### Effect of Liposome Encapsulated Peptides on Skin and Hair Growth in Humans

Nine patients with chronic psoriasis were enrolled in a double blind placebo controlled trial. Two comparable lesions received either PTH (1-34) formulated in Novasome at a concentration of 20 μg/0.1 ml and the contralateral lesion received 100 μl of Novasome A placebo for a period of two months. The percentage of clinical improvement of scaling, erythema, and induration for the lesion treated with placebo was 17.2, 15, and 30%, respectively. For the lesion treated with PTH (1-34), there was a marked improvement in scaling of 30.9%, erythema 34%, and induration 52%, respectively. An evaluation of the skin biopsies analyzed by hemotoxilin and eosin staining and by immunohistochemistry for proliferating cell nuclear antigen and transglutaminase demonstrated that the lesions treated with PTH (1-34) compared to the placebo treated lesions showed a marked decrease in the proliferation of the epidermis, restoration of the PCNA staining in the basal layer, and restoration of transglutaminase staining in the upper granular layer only of the lesions treated with PTH (1-34) compared to the lesion treated with placebo control Novasome. These data indicate that the topical application of PTH (1-34) in Novasome A restored psoriatic proliferation and differentiation to normal.

One patient with chronic psoriasis received topically PTH (1-34) for two months. He had a minimal response. The patient returned two weeks later and, on the lesion treated with PTH (1-34) and the placebo lesion, received topically calcipotriene cream. After two months of treatment, there was complete resolution of the psoriatic lesion that had previously received a topical application of PTH (1-34) for two months. There was no significant improvement in the lesion that received calcipotriene therapy and had received, previously, topical Novasome placebo. Therefore, the combination therapy of PTH (1-34) followed by calcipotriene or other activated vitamin D compounds can be an effective therapeutic approach for treating psoriasis.

### REFERENCES

(1) Schilli, M. B. Ray, S., Paus, R., Obi-Tabot, E. and Holick, M. F. Control of hair growth with parathyroid hormone (7-34). J. Invest. Dermatol. 108:928-932, 1997.
(2) Holick, M. F., Ray, S., Chen, T., Tian, X., and Persons, K. Novel finctions of a parathyroid hormone antagonist: stimulation of epidermal proliferation and hair growth in mice. Proc. Natl. Acad. Sci. 91:8014-8016,1994.
(3) Paus, R. and Cotsarelis, G. The biology of hair follicles. N. Engl. J. Med. 341: 491-497, 1999.
(4) Paus, R, Stenn, K. S., and Link, R. E. The induction of anagen hair growth in telogen mouse skin by cyclosporine A administration. Laboratory Invest. 60: 365-369, 1989.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of inhibiting proliferation or enhancing differentiation of a mammalian skin or hair cell, said method comprising topically administering to the mammalian skin or hair cell in need of inhibited proliferation or enhanced differentiation with a proliferation-inhibiting or differentiation-enhancing amount of a peptide or a salt or derivative thereof encapsulated within a liposome, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes, or in vivo in mouse skin by inhibiting skin cell proliferation or hair cycle progression or hair cell growth; wherein said liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

2. The method of claim 1, wherein said peptide is PTH (1-34), PTHrP (1-34), PTH (1-84), PTHrP (1-141), PTHrP (1-139) or PTHrP (1-173).

3. The method of claim 1, wherein said method is a method of inhibiting a hyperproliferative skin disorder.

4. The method of claim 3, wherein said hyperproliferative skin disorder is psoriasis, ichthyosis, actinic keratosis, or skin cancer.

5. The method of claim 1, wherein said method is a method of inhibiting hair growth or preventing hair regrowth.

6. The method of claim 1, wherein said peptide has at least 75% sequence identity with the 34 amino acid N-terminal region of hPTH or hPTHrP.

7. The method of claim 1, further comprising administering to the mammalian hair or skin cell an effective amount of an active vitamin D compound.

8. The method of claim 7, wherein said active vitamin D compound is calcipotriene.

9. The method of claim 7, wherein said peptide and active vitamin D compound are administered topically or parenterally.

10. A method of inhibiting proliferation or enhancing differentiation of a skin or hair cell of a mammal, said method comprising administering to the mammal in need thereof a proliferation-inhibiting or differentiation-enhancing amount of a peptide or a salt or derivative thereof and an active vitamin D compound, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes, or in vivo in mouse skin by inhibiting skin cell proliferation or hair cycle progression or hair cell growth.

11. The method of claim 10, wherein said peptide and said active vitamin D compound are administered as part of a single pharmaceutical composition.

12. The method of claim 10, wherein said peptide and said active vitamin D compound are administered as part of separate pharmaceutical compositions.

13. The method of claim 10, wherein said peptide is administered parentally.

14. The method of claim 10, wherein said active vitamin D compound is administered topically.

15. The method of claim 10, wherein said peptide is encapsulated within a liposome which comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

16. A method of inducing proliferation of a mammalian skin or hair cell, said method comprising topically administering to the mammalian skin or hair cell in need of proliferation with a proliferation-inducing amount of a peptide or a salt or derivative thereof encapsulated within a liposome, wherein the peptide is at least 3 amino acids long, has at least 10% sequence identity with the 34 amino acid N-terminal region of HPTH or hPTHrP, and is capable of blocking the inhibition of proliferation or stimulation of differentiation in vitro of cultured human keratinocytes by PTH (1-34), 1,25(OH)2D3 or PTHrP (1-34), or in vivo in mouse skin by stimulating skin cell proliferation or accelerating hair cycle progression or stimulating hair cell growth; wherein said liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

17. The method of claim 16, which is a method of stimulating skin cell growth, rejuvenating aged skin, preventing skin wrinkles, treating skin wrinkles, enhancing wound healing, stimulating hair growth, maintaining hair growth, treating or preventing female or male pattern baldness, or treating chemotherapy induced alopecia.

18. The method of claim 16, which is a method of stimulating epidermal cell growth or hair follicle cell growth.

19. The method of claim 16, wherein said peptide is PTH (7-34), PTHrP (7-34), PTH (5-36), PTHrP (5-36), PTH (5-34) or PTHrP (5-34).

20. A composition comprising a proliferation-inhibiting or differentiation-enhancing amount of a peptide or a salt or derivative thereof encapsulated within a liposome, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes, or in vivo in mouse skin by inhibiting skin cell proliferation or hair cycle progression or hair cell growth; wherein said liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-Cl₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

21. A composition comprising a proliferation-inducing amount of a peptide or a salt or derivative thereof encapsulated within a liposome, wherein the peptide is at least 3 amino acids long, has at least 10% sequence identity with the 34 amino acid N-terminal region of hPTH or hPTHrP, and is capable of blocking the inhibition of proliferation or stimulation of differentiation in vitro of cultured human keratinocytes by PTH (1-34), 1,25(OH)2D3 or PTHrP (1-34), or in vivo in mouse skin by stimulating skin cell proliferation or accelerating hair cycle progression or stimulating hair cell growth; wherein said liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.

22. A composition comprising a proliferation-inhibiting or differentiation-enhancing amount of a peptide or a salt or derivative thereof and an active vitamin D compound, wherein the peptide is at least 3 amino acids long, has at least 10% sequence identity with the 34 amino acid N-terminal region of hPTH or hPTHrP, and is capable of inhibiting proliferation or enhancing differentiation in vitro of cultured human keratinocytes, or in vivo in mouse skin by inhibiting skin cell proliferation or hair cycle progression or hair cell growth.

23. The composition of claim 22, wherein at least one of said peptide or active vitamin D compound is encapsulated by liposomes.

24. The composition of claim 23; wherein said liposome comprises at least two distinct lipids, a primary lipid and a secondary lipid, the primary lipid constituting the greatest proportion, by weight, of any single lipid material forming the bilayers of said vesicle, the primary lipid being selected from the group consisting of C₁₂-C₁₈ fatty alcohols, C,₁₂-C₁₈ glycol monoesters, C₁₂-C₁₈ gyceryl mono-and diesters, and mixtures thereof, and the primary lipid further having the property that it will to form a lipid vesicle in the absence of the secondary lipid, and the secondary lipid being present in an amount sufficient to allow formation of the lipid vesicles, the secondary lipid being selected from the group consisting of quaternary dimethyldiacyl amines, polyoxyethylene acyl alcohols, polyglycerols, sorbitan fatty acid esters, fatty acids and their salts, and mixtures thereof.
